(19) United States
(12) Patent Application Publication
(10) Pub. No.: US 2025/0290142 A1
(43) Pub. Date: Sep. 18, 2025

(54) DETECTING OXIDATIVE STRESS IN CELL(S) USING EPIGENETIC MEANS

(71) Applicant: Evonik Operations GmbH, Essen (DE)

(72) Inventors: Florian BÖHL, Neckargemund (DE); Suki ROY, Singapore (SG); Jennifer BOURLAND, Singapore (SG); Sanjanaa NAGARAJAN, Singapore (SG)

(73) Assignee: Evonik Operations GmbH, Essen (DE)

(21) Appl. No.: 18/861,943

(22) PCT Filed: Apr. 21, 2023
(86) PCT No.: PCT/EP2023/060491

§ 371 Date: Oct. 31, 2024

(30) Foreign Application Priority Data
May 3, 2022 (EP) 22171359.7

(51) Int. Cl. C12Q 1/6883; C12Q 1/6806

(52) U.S. Cl. CPC C12Q 1/6883; C12Q 1/6806; C12Q 2600/154

(57) ABSTRACT

The present invention is related to a method of identifying oxidative stress (OS) in a test cell, comprising
(a) determining the methylation status of at least one CpG site in a DNA sample obtained from the test cell,
(b) comparing the methylation status of the CpG site from (a) with that of a control without OS,
   wherein difference in the methylation status of the CpG site in the test cell compared to the CpG site in the control is indicative of the test cell having OS.

### FIELD OF THE INVENTION

The present invention relates to a method for detecting oxidative stress (OS) in a cell using epigenetic markers. In particular, the method is capable of identifying OS in cell by determining the methylation status of a CpG site in a test cell and comparing the resultant methylation status with a reference methylation status of a control cell without OS. Differential methylation of the CpG site in the test cell indicates that the test cell has OS.

### BACKGROUND OF THE INVENTION

Living organisms are subjected continuously to a variety of stresses from the outside environment. In order to resist such stresses, they maintain their homeostasis by various regulatory systems. Oxidative stress refers to a serious imbalance between the levels of reactive oxygen species (ROS) in a cell and its antioxidant defense mechanism. In order to survive this stress, living organisms have a system called redox regulation to cope with the stress to maintain their homeostasis by regulating the redox state. This system functions to adapt to many external stress agents such as radiation, ultraviolet (UV) rays, environmental pollutants, high fever, low temperature, hypoxic condition, and infectious diseases as well as to oxidative stress from lifestyle-related diseases such as cancer, diabetes, arteriosclerosis, hypertension and obesity. However, if this regulation mechanism is broken for some reason or other, oxidative stress (OS) occurs. OS can lead to cellular damage, DNA fragmentation, apoptosis and cell death. Early detection of OS can prevent further damage in the living organism causing the organism to receive early treatment or start using protection.

There are some methods known in the art for early detection of OS. However, none of these methods known in the art have been officially used to detect OS in a cell by using a genomic sample of the body.

The human skin is constantly exposed to oxidative stress and free radicals, such as to high quantities of ROS, derived not only from ordinary metabolic reactions but also continuous exposure to air, radiation and UV rays, environmental pollutants, as well as physical and/or chemical agents (e.g., cosmetics). Under some conditions, the production of ROS may become so great that is may contribute to the pathogenesis of, for example, psoriasis or skin cancer. Oxidative damage caused by free radicals such as ROS is also a main cause of physical ageing in general, and of the skin in particular. Accordingly, there is a need in the art for detection of OS in cells, for example skin cells to prevent further damage to the cells.

There are some methods known in the art for early detection of OS. However, none of these methods known in the art have been officially used to detect OS in a cell by using a genomic sample of the body.

The human skin is constantly exposed to oxidative stress and free radicals, such as to high quantities of ROS, derived not only from ordinary metabolic reactions but also continuous exposure to air, radiation and UV rays, environmental pollutants, as well as physical and/or chemical agents (e.g., cosmetics). Under some conditions, the production of ROS may become so great that it may contribute to the pathogenesis of, for example, psoriasis or skin cancer. Oxidative damage caused by free radicals such as ROS is also a main cause of physical ageing in general, and of the skin in particular. Accordingly, there is a need in the art for detection of OS in cells, for example skin cells to prevent further damage to the cells.

### DESCRIPTION OF THE INVENTION

The present invention attempts to solve the problems above by providing a method of detecting Oxidative Stress (OS) in a test cell by comparing the methylation status of at least one CpG site in the test cell and the corresponding CpG site in a control cell with no OS, wherein the presence of hypomethylation or hypermethylation at the CpG site in the test cell is indicative of the test cell having OS.

Since environmental factors/agents such as, UV light exposure, ageing, diet and the like, may trigger OS which can further induce an alteration in the promoter CpG methylation status of the gene by recruiting DNA methyltransferases (DNMTs) and TET enzymes to various promoters, biomarkers that result in differential methylation in a cell with OS is essential to overcome the problems mentioned above. In particular, CpG sites can be used as biomarkers for detecting OS in a cell. CpG sites in a cell with OS are differentially methylated (i.e. hypomethylated or hypermethylated) compared to the corresponding CpG sites in a cell without OS. Accordingly, these CpG sites may be effectively used to determine if a cell has OS. This is particularly advantageous as using epigenetics provides a means of predicting the onset of OS in a cell, thus allowing OS to be treated earlier before causing even more damage to the cell. Further, an epigenetic marker is a long-term biomarker, that is to say it is inheritable and can be used to detect OS in the next generation as well if need be.

According to one aspect of the present invention, there is provided a method of identifying oxidative stress (OS) in a test cell, comprising
(a) determining the methylation status of at least one CpG site in a DNA sample obtained from the test cell,
(b) comparing the methylation status of the CpG site from (a) with that of a control without OS,
   wherein difference in the methylation status of the CpG site in the test cell compared to the CpG site in the control is indicative of the test cell having OS.

As used herein, the term “cell” refers to an intact live cell, naturally occurring or modified. The cell may be isolated from other cells, mixed with other cells in a culture, or within a tissue (partial or intact), or an organism. In particular, the cell may be a eukaryote cell. More in particular, the cell may be mammalian cell. The term “mammalian cell” refers to any cell derived from a mammalian subject. The cell may also be a cell derived from the culture and expansion of a cell obtained from a subject. The cell may also have been genetically modified to express a recombinant protein and/or nucleic acid. The mammalian cell may be from humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In particular, the subject is a mammal. More in particular, the mammal is selected from the group consisting of a mouse, a rat, a guinea pig, a dog, a mini-pig, a human being, a cow, a sheep, a pig, a goat, a horse, a donkey, and a mule. In particular, the mammalian cell may be a skin cell, a stem cell or a cell derived therefrom. More in particular, the mammalian cell may be a skin cell.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell with OS compared to a cell with no OS.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG-and CpNpN-sequences may be considered the most common. In principle, other naturally occurring nucleotides may also be methylated but they will not be taken into consideration with regard to any aspect of the present invention.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to a cell with no indication of OS.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to a cell with no indication of OS.

As used herein, the term “genomic material” refers to nucleic acid molecules or fragments of the genome of the subject or group of subjects. In particular, such nucleic acid molecules or fragments are DNA or RNA or hybrids thereof, and most preferably are molecules of the DNA genome of a subject or group of subjects.

As used herein, the “DNA sample” refers to the DNA extracted from the cell according to any aspect of the present invention using known methods in the art.

In particular, when there is differential methylation detected in a test cell, that is to say that the cell displays hypermethylation or hypomethylation at, at least one CpG site in comparison to the control (i.e., a cell without indication of OS), then the test cell has OS. More in particular, when the CpG site displays hypomethylation in the test cell in comparison to the corresponding CpG site in the control cell, the test cell has OS. In another example, when the CpG site displays hypermethylation in the test cell in comparison to the corresponding CpG site in the control cell, the test cell has OS.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

More in particular, in step (a) the CpG site is selected from the list provided in Table 2:

TABLE 2
list of 96 differentially methylated CpG sites in OS
Probe ID, chromosome, position

cg10435849, chr21, 47518747
cg11349857, chr19, 2388952
cg00848693, chr16, 69400894
cg21149107, chr14, 71228406
cg02585025, chr16, 74347151
cg18693345, chr5, 2754148
cg25126321, chr11, 66462313
cg22155376, chr11, 17591837
cg17251097, chr10, 81838583
cg16098051, chr17, 39663178
cg23153556, chr11, 64900163
cg11271430, chr4, 187984718
cg22545649, chr4, 96470459
cg05736120, chr6, 170125165
cg14417917, chr9, 140065568
cg23526353, chr4, 100484905
cg02884346, chr4, 6271501
cg12528713, chr4, 81187605
cg05943476, chr2, 70780904
cg03379894, chr5, 141131868
cg17135325, chr3, 160939158
cg16362899, chr10, 121554998
cg25546329, chr19, 40175243
cg00532451, chr1, 228528913
cg02339482, chr3, 191068318
cg01955286, chr3, 125475060
cg23268879, chr7, 5359951
cg01944087, chr15, 69110737
cg06544141, chr1, 27320384
cg20076186, chr3, 128372563
cg20906710, chr12, 50065474
cg25319564, chr13, 114214573
cg21598631, chr2, 242154679
cg20393002, chr3, 120909792
cg27626141, chr8, 103876469
cg14075645, chr21, 47552343
cg08351474, chr20, 4666916
cg15047751, chr3, 52395045
cg23247704, chr1, 116518985
cg05791356, chr3, 11578196
cg01728507, chr4, 3563357
cg12833948, chr16, 1384648
cg08714407, chr2, 24307071
cg18908499, chr1, 247712237
cg15709169, chr12, 125549783
cg12383788, chr1, 22471748
cg05831191, chr16, 67201012
cg06818207, chr6, 6003238
cg23441673, chr11, 134831429
cg11785465, chr1, 51717051
cg01429408, chr1, 156860314
cg02075410, chr4, 151504138
cg17773376, chr3, 107941759
cg12365107, chr3, 52720035
cg23023126, chr19, 308808
cg08721076, chr2, 172967670
cg09325695, chr14, 37825967
cg11434693, chr1, 226349343
cg04110544, chr5, 42424942
cg07758428, chr11, 68193549
cg10811474, chr19, 8428787
cg23807890, chr2, 228736357
cg16112558, chr17, 18157478
cg14751914, chr18, 46477404
cg10853608, chr16, 713101
cg11986082, chr2, 66652819
cg12271199, chr1, 59248280
cg25458727, chr3, 10261761
cg14418275, chr11, 66243281
cg19936912, chr4, 152330012
cg02318866, chr17, 74723064
cg18129198, chr1, 63249693
cg24637319, chr11, 88071195
cg09332835, chr11, 784136
cg22425466, chr9, 140115987
cg18117149, chr6, 41746516
cg15268826, chr4, 132897691
cg23636099, chr2, 114359251
cg09788788, chr4, 87468529
cg26163843, chr14, 34944819
cg13864229, chr14, 37051830
cg17942925, chr5, 180611441
cg18659937, chr11, 66669653
cg03611733, chr15, 45585637
cg13514165, chr9, 130544369
cg04360557, chr1, 33282743
cg11578055, chr4, 10447440
cg01283246, chr5, 135266135
cg21606780, chr1, 197881327
cg09009011, chr9, 117030773
cg11926460, chr2, 234378272
cg11718315, chr15, 65579480
cg22054008, chr9, 131873921
cg07896133, chr16, 2060022
cg25109721, chr17, 48125064
cg20671801, chr12, 51041328

The method according to any aspect of the present invention, further comprises the step of:
(i) performing bisulfite modification to the DNA sample before step (a).

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37:424-429 (2019).

The cell used according to any aspect of the present invention is obtained from a biological sample selected from the group consisting of blood, brain, sperm and any other tissue or sample that provides genomic DNA to be used in the method according to any aspect of the present invention. In particular, the biological sample may comprise any biological material obtained from the subject that contains DNA, and may be liquid, solid or both, may be tissue or bone, or a body fluid such as blood, lymph, etc. In particular, the biological sample useful for the present invention may comprise biological cells or fragments thereof.

The term “test” used in conjunction with the term cell herein refers to a cell that is subjected to the method according to any aspect of the present invention and is the basis for an analysis application of the present invention. A ‘test cell’ is therefore a cell or a group of cells being tested according to any aspect of the present invention or a profile being obtained or generated in this context. Conversely, the term “reference” or ‘control’ shall denote, mostly predetermined, entities which are used for a comparison with the test entity. In particular, a ‘test cell’ refers to a cell being tested for OS where the methylation status has to be determined and a ‘control’ refers to a cell without OS where the methylation status is already known and used as a reference.

The OS according to any aspect of a the present invention may be caused or may be a result of UV light exposure, ageing, H₂O₂ exposure and a combination thereof (i.e. UV light and H₂O₂ exposure, UV light exposure and ageing, H₂O₂ exposure and ageing or UV light and H₂O₂ exposure and ageing.

According to a further aspect of the present invention, there is provided a method of detecting the incidence of oxidative stress (OS) in a cell, the method comprising detecting an epigenetic change in at least one CpG site in the cell, wherein detection of the epigenetic change is indicative of the incidence of OS and wherein the epigenetic change is methylation.

In particular, the methylation is hypomethylation. DNA hypomethylation profiling may be very useful for stratifying cell cultures systems ranging from 1D to 3D, stem cells to differentiated skin tissue models under stress.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

### BRIEF DESCRIPTION OF FIGURES

FIG. 1A is a scatter plot showing that a large number of probes have a different methylation status in cell with where artificial OS (high UV for 24 hrs) was induced according to Example 1. As can be seen, there is an equally large number of probes that are hypomethylated as there are probes hypermethylated.

FIG. 1B is a box-plot confirming the results in FIG. 1A that a large number of probes have a different methylation status in cell with where artificial OS (high UV for 24 hrs) was induced according to Example 1.

FIG. 2A is a scatter plot showing that a large number of probes have a different methylation status in cell with where artificial OS (low UV for 72 hrs) was induced according to Example 1. As can be seen, there is an equally large number of probes that are hypomethylated as there are probes hypermethylated.

FIG. 2B is a box-plot confirming the results in FIG. 2A that a large number of probes have a different methylation status in cell with where artificial OS (low UV for 72 hrs) was induced according to Example 1.

FIG. 3A is a scatter plot showing that a large number of probes have a different methylation status in cell with where artificial OS (low H₂O₂ for 24 hrs) was induced according to Example 2. As can be seen, there is a large number of probes that are hypomethylated in cells with OS compared to cells without OS.

FIG. 3B is a box-plot confirming the results in FIG. 3A that a large number of probes have a different methylation status in cell with where artificial OS (low H₂O₂ for 24 hrs) was induced according to Example 2.

FIG. 4A is a scatter plot showing that a large number of probes have a different methylation status in cell with where artificial OS (high H₂O₂ for 24 hrs) was induced according to Example 2. As can be seen, there is a large number of probes that are hypomethylated in cells with OS compared to cells without OS.

FIG. 4B is a box-plot confirming the results in FIG. 4A that a large number of probes have a different methylation status in cell with where artificial OS (high H₂O₂ for 24 hrs) was induced according to Example 2.

FIG. 5A is a scatter plot showing that a large number of probes have a different methylation status in cell with where artificial OS according to Example 3 with Medox® in cells was induced. As can be seen, there is a large number of probes that are hypomethylated in cells with OS compared to cells without OS.

FIG. 5B is a box-plot confirming the results in FIG. 5A that a large number of probes have a different methylation status in cell with where artificial OS according to Example 3 with Medox® in cells was induced.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Oxidative Stress on Human Tissue With UV

Artificial oxidative stress was induced in the cell culture system and skin tissue model to analyze the methylation status of promoters.

T-Skin models were obtained from Episkin SA, France which is composed of reconstructed human skin. Each skin model consists of a dermal equivalent overlaid by a stratified, well-differentiated epidermis derived from normal human keratinocytes. Upon receiving the skin models, it was recovered by incubating in T-Skin culture medium overnight at 37° C. in a 5% CO₂ incubator.

To induce oxidative stress on a human tissue system, skin models (5x replicates) were exposed to UV radiation (UVA 24 J/cm²+UVB 50 mJ/cm²) (high UV) daily for 24 hrs.

In another group, skin models (5x replicates) were exposed to UV radiation (UVA 12 J/cm²+UVB 25 mJ/cm²daily) (low UV) daily for 72 hrs.

Exposure to UV radiation leads to the generation of ROS which finally results in the development of oxidative stress within the cells. A control set of skin models (5x replicates) were maintained for 72 hrs without any exposure to UV radiation. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Quality Control and Data Processing

Methylation EPIC array data processing was performed in R version 4.1.2 (2021 Nov. 1) using the minfi version 1.40.0. The raw intensity data (IDAT) were imported into the R (4.1.2), processed using the minfi (1.4.0) Bioconductor package, 18. Quality check on samples was performed to keep probes that have a detection P-value <0.01 in one or more samples or have a mean detection P-value <0.05 in all samples. Then samples were normalized using functional normalization (implemented by preprocessFunnorm function in minfi) for type-bias correction and background correction.

Prior to differential methylation analysis, the probes with non-specific binding, cross reactive probes, probes affected by common SNPs, and probes annotated to the X, Y chromosomes were also filtered out. Beta-value and M-value of normalized and filtered samples were calculated using getBeta and getM function respectively, the samples were then subjected to further downstream analysis.

### Differential Methylation Analysis

Differential methylation analysis was performed using packages limma version 3.50.1 and DMRcate version 2.8.5. Contrast matrix was set up by comparing each corresponding treatment and control group and empirical Bayesian algorithm was used to fit the M-values based on the design and contrast model. Probes with adjusted P-value lower than 0.05 were considered as differentially methylation positions (DMPs). Annotation was performed using IlluminaHumanMethylationEPICkanno.ilmn12.hg19 and annotatr package (1.20.0).

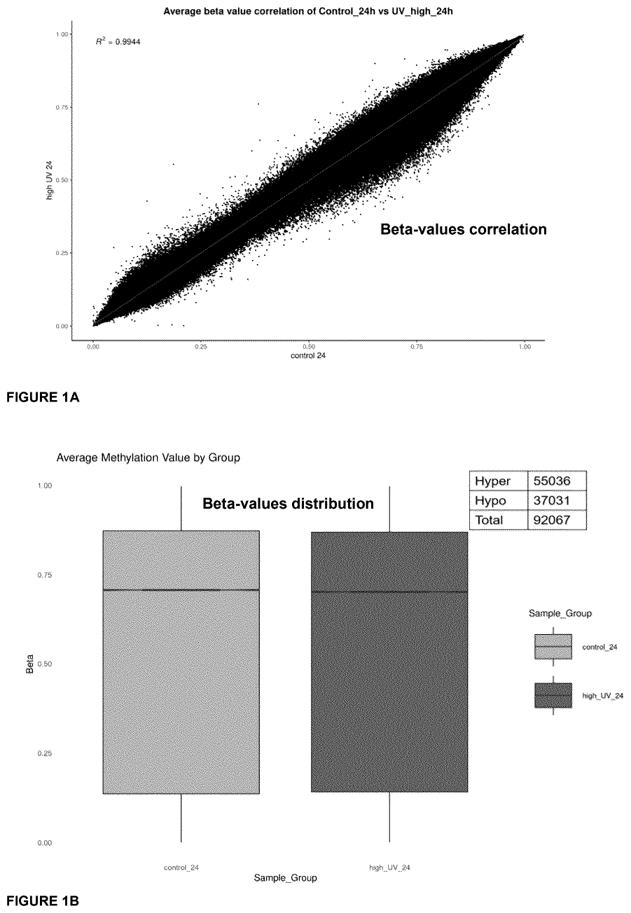

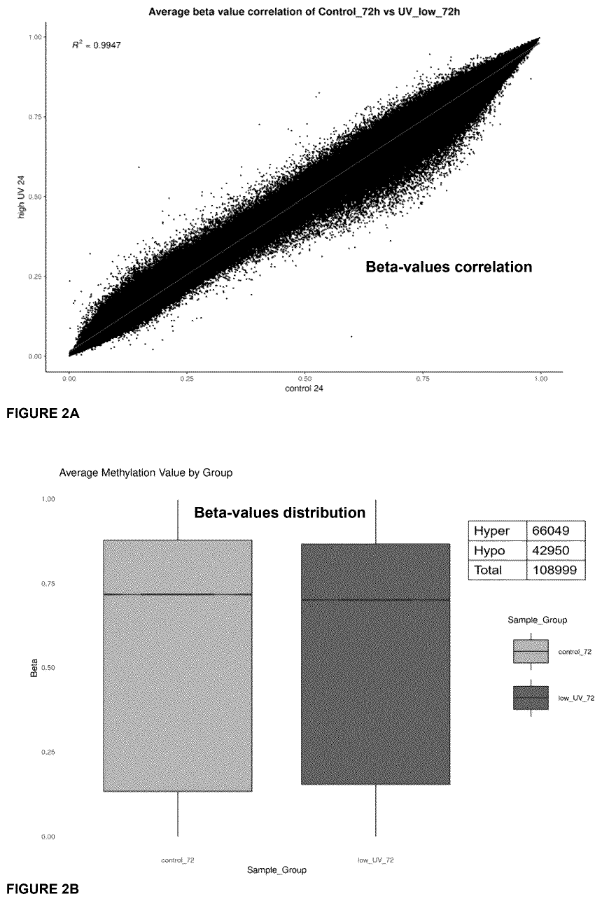

As seen in FIGS. 1A and B and 2A and 2B there are many probes that were differentially methylated in the cell with OS compared to a control cell with no OS. The results also show that there were equally as many hypomethylated probes as hypermethylated probes.

### Example 2

Oxidative Stress on Human Tissue With H₂O₂

Another way to induce oxidative stress on a human tissue system is through Hydrogen peroxide treatment which leads to the generation of ROS within the cells. The skin models (5x replicates) were treated with two different concentrations of Hydrogen peroxide [100 μM (low) and 200 μM (high)] for 2 hrs and were maintained for 24 hrs. A control set of skin models (5x replicates) were maintained for 24 hrs without any treatment with Hydrogen peroxide. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

Same method of quality control and data processing as that disclosed in Example 1 was carried out on the samples here. Further, the same differential methylation analysis as disclosed in Example 1 was carried out on the data obtained from Example 2.

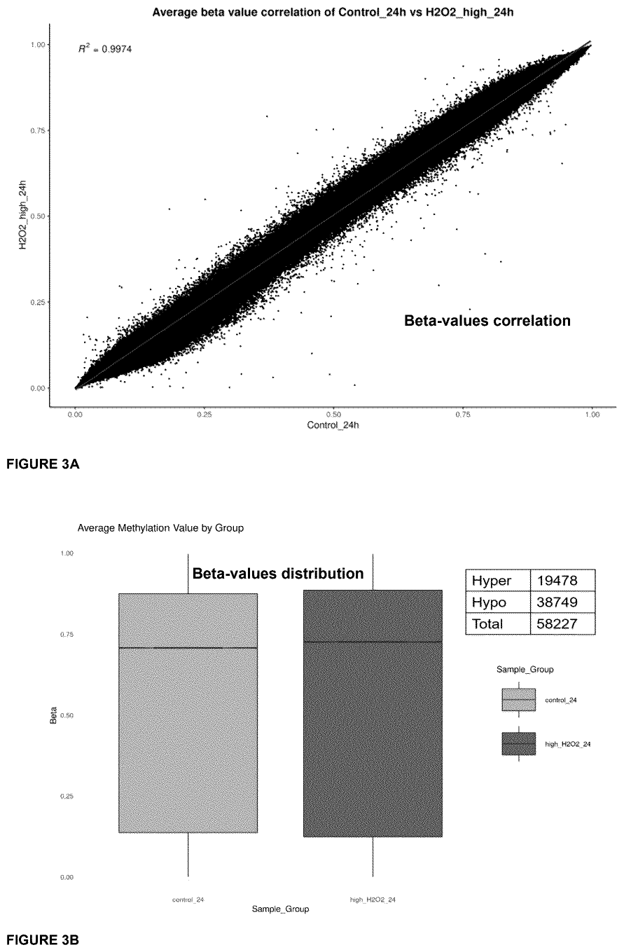

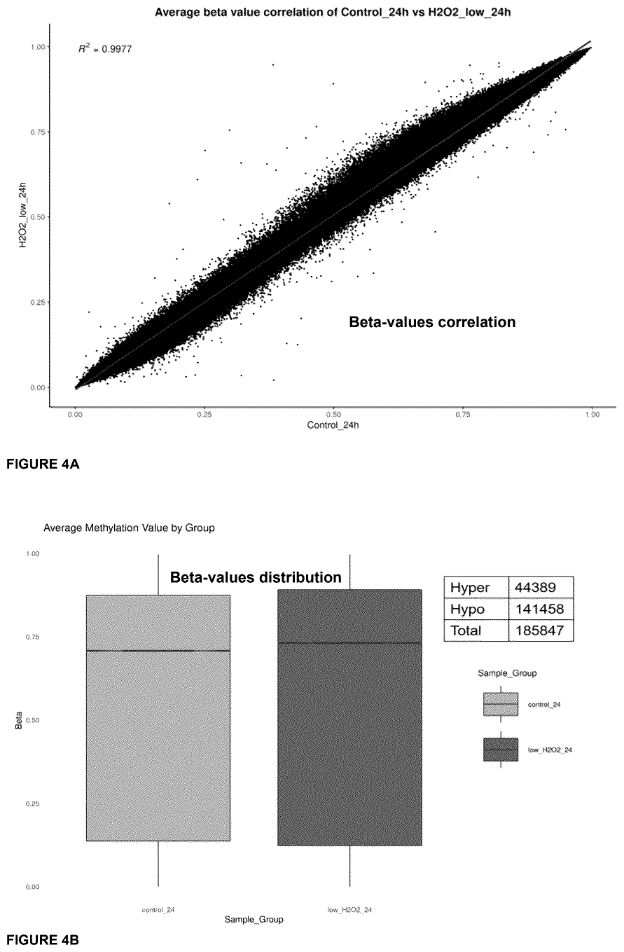

As seen in FIGS. 3A and 3B and 4A and 4B, there are many probes that were differentially methylated in the cell with OS compared to a control cell with no OS. There is a large number of probes that are hypomethylated in cells with OS compared to cells without OS.

### Example 3

### Oxidative Stress on Cell Culture System With Medox®

To investigate oxidative stress in the cell culture system, Mesenchymal Stem Cells (MSCs) were treated with Medox (Evonik, Batch: H-080719) which contains a lot of natural anthocyanins, associated with antioxidative and anti-inflammatory properties. Bone marrow derived MSCs were cultured for 1 week in Mesencult ACF Plus Medium with two doses of Medox (4x replicates): 25 μg/ml (low) and 100 μg/ml (high). The media with Medox was replaced every second day for 1 week. As a control (4x replicates), MSCs were cultured for 1 week in Mesencult ACF Plus Medium without any Medox® treatment. Medox® treatment is expected to produce the opposite reaction to OS.

This was followed by collection of cell pellet and genomic DNA was purified from the cell pellet using the DNeasy® Blood & Tissue Kit (Qiagen). The genomic DNA was quantified using the PicroGreen® or NanoDrop™ 2000.

The genomic DNA (500 ng) from the cell pellet was subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

DNA methylation profiling has been proven to be a powerful analytical tool to accurately identify the origin of tissue and the effect of environmental factors. It has several advantages as a biomarker classifier as it is a stable marker, and it can facilitate quantitative analysis at single-nucleotide resolution.

Same method of quality control and data processing as that disclosed in Example 1 was carried out on the samples here. Further, the same differential methylation analysis as disclosed in Example 1 was carried out on the data obtained from Example 3.

With the low treatment to Medox® on MSCs, 35,532 differentially methylated probes (p<0.05) were identified, out of which 15,368 probes were hypermethylated and 20,164 probes were hypomethylated.

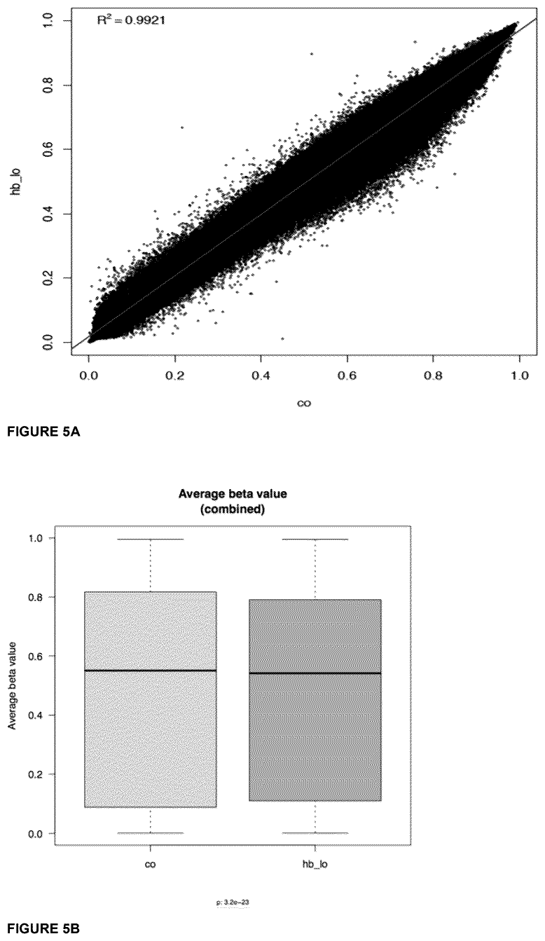

As seen in FIGS. 5A and B there are many probes that were differentially methylated in the cell with OS compared to a control cell with no OS. There is a large number of probes that are hypomethylated in cells with OS compared to cells without OS.

### Example 4

Oxidative Stress on Human Tissue With UV rays

Artificial oxidative stress was induced in the cell culture system and skin tissue model to analyze the methylation status of promoters.

T-Skin models were obtained from Episkin SA, France which is composed of reconstructed human skin. Each skin model consists of a dermal equivalent overlaid by a stratified, well-differentiated epidermis derived from normal human keratinocytes. Upon receiving the skin models, it was recovered by incubating in T-Skin culture medium overnight at 37° C. in a 5% CO₂ incubator.

To induce oxidative stress on a human tissue system, skin models (5x replicates) were exposed to UV radiation (UVA 24 J/cm²+UVB 50 mJ/cm²) (high UV) and cultured for 24 hrs.

In another group, skin models (5x replicates) were exposed to UV radiation (UVA 12 J/cm²+UVB 25 mJ/cm²daily) (low UV) and cultured for 24 hrs.

Exposure to UV radiation leads to the generation of ROS which finally results in the development of oxidative stress within the cells. A control set of skin models (5x replicates) were maintained for 24 hrs without any exposure to UV radiation. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

Oxidative Stress on Human Tissue With H₂O₂

Another way to induce oxidative stress on a human tissue system is through Hydrogen peroxide treatment which leads to the generation of ROS within the cells. The skin models (5x replicates) were treated with two different concentrations of Hydrogen peroxide [100 μM (low) and 200 μM (high)] for 2 hrs and were maintained for 24 hrs. A control set of skin models (5x replicates) were maintained for 24 hrs without any treatment with Hydrogen peroxide. Followed by the treatment, skin models were collected, and genomic DNA was purified from the tissue samples using the

DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Oxidative Stress on Human Tissue With Particulate Matter 2.5

Another way to induce oxidative stress on a human tissue system is through Particulate Matter 2.5 (PM2.5) treatment which leads to the generation of ROS within the cells by its chemical components and metals. The skin models (5x replicates) were treated with two different concentrations of PM2.5 [15 μg/cm²(low) and 30 μg/cm²(high)] and were maintained for 24 hrs. A control set of skin models (5x replicates) were maintained for 24 hrs without any treatment with PM2.5. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Oxidative Stress on Human Tissue With Glyoxal

Another way to induce oxidative stress on a human tissue system is through Glyoxal treatment which provokes oxidative stress by increasing the level of ROS within the cells by producing advanced glycation end-products. The skin models (5x replicates) were treated with two different concentrations of glyoxal [0.5 mM (low) and 1 mM (high)] and were maintained for 24 hrs. A control set of skin models (5x replicates) were maintained for 24 hrs without any treatment with glyoxal. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Oxidative Stress on Human Tissue With Ageing

Another way to induce oxidative stress on a human tissue system is through ageing which leads to the generation of Reactive oxygen and nitrogen species (RONS) within the cells.

The skin models were maintained in the deep well plate with media 14 days (6x replicates) with media being renewed after 7 days to induce ageing in the skin tissue. Skin models were collected after 14 days, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Quality Control and Data Processing

A total of 103 samples from 16 different treatments and their respective controls were analyzed. The treatments can be grouped into two batches. Batch 1 consisted of all 24 hour treatments, at high and low concentrations. All treatments in batch 1 shared the same control group. Table 1 shows the sample information for batch 1.

Methylation EPIC array data processing was performed in R version 4.2.2 (2021 Nov. 10 r83330) using the minfi version 1.42.0. The raw intensity data (IDAT) were imported into the R (4.2.2), processed using the minfi (1.42.0) Bioconductor package. Quality check on samples were performed to keep probes that had a detection P-value <0.01 in one or more samples or had a mean detection P-value <0.05 in all samples. The samples were then normalized using functional normalization (implemented by preprocesssFunnorm function in minfi) for type-bias correction and background correction.

Prior to differential methylation analysis, the probes with non-specific binding, cross reactive probes, probes affected by common SNPs, and probes annotated to the X, Y chromosomes were also filtered out. Beta-value and M-value of normalized and filtered samples were calculated using getBeta and getM function respectively, the samples were then subjected to further downstream analysis.

TABLE 1
Sample information for batch 1

| Group | Treatment | Replicates |
|---|---|---|
| Control | Untreated, 24 h | 5 |
| Pollution | Treated with 30 μg/cm² particulate matter 2.5 | 5 |
| Treatment | for 24 h | |
| | Treated with 15 μg/cm² particulate matter 2.5 | 5 |
| | for 24 h | |
| H₂O₂ | Treated with systemic exposure to 200 μM | 5 |
| Treatment | H₂O₂ for 2 h and cultured for 24 h | |
| | Treated with systemic exposure to 100 μM | 5 |
| | H₂O₂ for 2 h and cultured for 24 h | |
| Glyoxal | Treated with systemic exposure to 1 mM | 5 |
| Treatment | glyoxal 1 mM for 24 h | |
| | Treated with systemic exposure to 0.5 mM | 5 |
| | glyoxal 1 mM for 24 h | |
| UV-radiation | Exposed to UVA 24 J/cm2 + UVB 50 mJ/cm2 | 5 |
| Treatment | and cultured for 24 h | |
| | Exposed to UVA 12 J/cm2 + UVB 25 mJ/cm2 | 5 |
| | and cultured for 24 h | |
| Aged | Untreated, cultured for 2 weeks in media | 6 |

### Differential Methylation Analysis

Pair-wise differential methylation analysis (total of 16 pairs) was performed using the limma package version 3.52.4. The batch 1 samples were analyzed together. Contrast matrix was set up by comparing each corresponding treatment and control group and empirical Bayesian algorithm was used to fit the M-values based on the design and contrast model. Probes with adjusted P-value lower than 0.05 were considered as differentially methylation positions (DMPs). After which, the DMPS within the batch 1 comparisons, batch 1 High concentration comparisons, and batch 1 Low concentration comparisons were compared to identify common DMPs that are present in all comparisons within each group and have the same methylation status throughout.

Batch 1 comparisons had a total of 96 common DMPs, Batch 1 High comparisons had a total of 616 common DMPs and Batch 1 Low comparisons had a total of 238 common DMPs. Tables 2,3 and 4 show the list of common DMPs within each group for Batch 1 comparisons, Batch 1 High comparisons, and Batch 1 Low comparisons respectively.

TABLE 2
List of common DMPs within group for
Batch 1 comparisons (i.e. all treatments)
Probe ID, chromosome, position

cg10435849, chr21, 47518747
cg11349857, chr19, 2388952
cg00848693, chr16, 69400894
cg21149107, chr14, 71228406
cg02585025, chr16, 74347151
cg18693345, chr5, 2754148
cg25126321, chr11, 66462313
cg22155376, chr11, 17591837
cg17251097, chr10, 81838583
cg16098051, chr17, 39663178
cg23153556, chr11, 64900163
cg11271430, chr4, 187984718
cg22545649, chr4, 96470459
cg05736120, chr6, 170125165
cg14417917, chr9, 140065568
cg23526353, chr4, 100484905
cg02884346, chr4, 6271501
cg12528713, chr4, 81187605
cg05943476, chr2, 70780904
cg03379894, chr5, 141131868
cg17135325, chr3, 160939158
cg16362899, chr10, 121554998
cg25546329, chr19, 40175243
cg00532451, chr1, 228528913
cg02339482, chr3, 191068318
cg01955286, chr3, 125475060
cg23268879, chr7, 5359951
cg01944087, chr15, 69110737
cg06544141, chr1, 27320384
cg20076186, chr3, 128372563
cg20906710, chr12, 50065474
cg25319564, chr13, 114214573
cg21598631, chr2, 242154679
cg20393002, chr3, 120909792
cg27626141, chr8, 103876469
cg14075645, chr21, 47552343
cg08351474, chr20, 4666916
cg15047751, chr3, 52395045
cg23247704, chr1, 116518985
cg05791356, chr3, 11578196
cg01728507, chr4, 3563357
cg12833948, chr16, 1384648
cg08714407, chr2, 24307071
cg18908499, chr1, 247712237
cg15709169, chr12, 125549783
cg12383788, chr1, 22471748
cg05831191, chr16, 67201012
cg06818207, chr6, 6003238
cg23441673, chr11, 134831429
cg11785465, chr1, 51717051
cg01429408, chr1, 156860314
cg02075410, chr4, 151504138
cg17773376, chr3, 107941759
cg12365107, chr3, 52720035
cg23023126, chr19, 308808
cg08721076, chr2, 172967670
cg09325695, chr14, 37825967
cg11434693, chr1, 226349343
cg04110544, chr5, 42424942
cg07758428, chr11, 68193549
cg10811474, chr19, 8428787
cg23807890, chr2, 228736357
cg16112558, chr17, 18157478
cg14751914, chr18, 46477404
cg10853608, chr16, 713101
cg11986082, chr2, 66652819
cg12271199, chr1, 59248280
cg25458727, chr3, 10261761
cg14418275, chr11, 66243281
cg19936912, chr4, 152330012
cg02318866, chr17, 74723064
cg18129198, chr1, 63249693
cg24637319, chr11, 88071195
cg09332835, chr11, 784136
cg22425466, chr9, 140115987
cg18117149, chr6, 41746516
cg15268826, chr4, 132897691
cg23636099, chr2, 114359251
cg09788788, chr4, 87468529
cg26163843, chr14, 34944819
cg13864229, chr14, 37051830
cg17942925, chr5, 180611441
cg18659937, chr11, 66669653
cg03611733, chr15, 45585637
cg13514165, chr9, 130544369
cg04360557, chr1, 33282743
cg11578055, chr4, 10447440
cg01283246, chr5, 135266135
cg21606780, chr1, 197881327
cg09009011, chr9, 117030773
cg11926460, chr2, 234378272
cg11718315, chr15, 65579480
cg22054008, chr9, 131873921
cg07896133, chr16, 2060022
cg25109721, chr17, 48125064
cg20671801, chr12, 51041328

TABLE 3
List of common DMPs within group for Batch 1 high comparisons
Probe ID, chromosome, position

cg10435849, chr21, 47518747
cg04542433, chr16, 66460861
cg08524698, chr22, 21213167
cg02759622, chr19, 4831885
cg11349857, chr19, 2388952
cg03297966, chr4, 77818419
cg00848693, chr16, 69400894
cg21149107, chr14, 71228406
cg08521332, chr16, 33965573
cg02585025, chr16, 74347151
cg26071963, chr14, 88945568
cg18761773, chr12, 56498536
cg13544851, chr7, 154002372
cg11807318, chr7, 2566252
cg19705215, chr14, 61747501
cg17177084, chr14, 100604840
cg18693345, chr5, 2754148
cg25317268, chr10, 134258313
cg12405421, chr1, 35222918
cg25126321, chr11, 66462313
cg04702766, chr16, 70435106
cg08521046, chr2, 86362005
cg01406836, chr13, 42145979
cg22155376, chr11, 17591837
cg15212440, chr10, 104474465
cg17637107, chr8, 20054583
cg20251110, chr12, 124813301
cg10842775, chr6, 21761442
cg08806153, chr2, 176971961
cg04283403, chr6, 150326126
cg17251097, chr10, 81838583
cg16879432, chr19, 33786219
cg15641162, chr4, 185942800
cg12350116, chr12, 110035956
cg16098051, chr17, 39663178
cg18155210, chr20, 3223112
cg27401784, chr1, 46974443
cg05718255, chr22, 35790141
cg19641582, chr12, 71003638
cg07146974, chr7, 51275251
cg25546329, chr19, 40175243
cg24119722, chr22, 42347272
cg09194750, chr11, 34937341
cg00532451, chr1, 228528913
cg02339482, chr3, 191068318
cg08351474, chr20, 4666916
cg15047751, chr3, 52395045
cg27297626, chr2, 23785055
cg18895813, chr21, 28339705
cg03609398, chr8, 145913076
cg22879676, chr17, 81009871
cg09469984, chr1, 9796095
cg08714407, chr2, 24307071
cg01746550, chr2, 60543791
cg18908499, chr1, 247712237
cg17287998, chr5, 59711350
cg02363737, chr6, 116210122
cg11174851, chr11, 69634592
cg11785465, chr1, 51717051
cg27570984, chr16, 55125644
cg01429408, chr1, 156860314
cg15772361, chr18, 61328223
cg15822010, chr7, 206484
cg20122548, chr15, 45694411
cg17022540, chr1, 2144214
cg05782361, chr2, 54773839
cg23153556, chr11, 64900163
cg19254558, chr3, 183602222
cg06905970, chr19, 41222748
cg11271430, chr4, 187984718
cg22545649, chr4, 96470459
cg04454086, chr5, 140345966
cg09253914, chr2, 37311580
cg02948541, chr16, 16427335
cg04964471, chr12, 54769641
cg05736120, chr6, 170125165
cg14010550, chr19, 1009642
cg16454084, chr6, 58147041
cg11032208, chr5, 95997736
cg12277998, chr20, 33999883
cg23631636, chr2, 217363620
cg14417917, chr9, 140065568
cg23526353, chr4, 100484905
cg13715127, chr17, 72856825
cg11313080, chr15, 38794891
cg02884346, chr4, 6271501
cg12528713, chr4, 81187605
cg05943476, chr2, 70780904
cg14462321, chr9, 137232372
cg03379894, chr5, 141131868
cg20326928, chr1, 16399560
cg13573073, chr12, 8940075
cg25307468, chr19, 3604804
cg17135325, chr3, 160939158
cg07573061, chr13, 32420690
cg16362899, chr10, 121554998
cg01485266, chr19, 2476132
cg04862885, chr11, 1248653
cg04747007, chr19, 7585845
cg01955286, chr3, 125475060
cg23268879, chr7, 5359951
cg21334654, chr2, 178128211
cg24078065, chr21, 43372534
cg12330281, chr1, 200708527
cg19412946, chr14, 81637075
cg03982350, chr16, 87184047
cg02264567, chr1, 156863732
cg00681792, chr19, 55677269
cg17341676, chr20, 43991995
cg01973174, chr1, 3441168
cg01944087, chr15, 69110737
cg06544141, chr1, 27320384
cg20076186, chr3, 128372563
cg20906710, chr12, 50065474
cg22253436, chr8, 140659783
cg01528287, chr6, 108010793
cg13999880, chr12, 25802509
cg25319564, chr13, 114214573
cg21598631, chr2, 242154679
cg23675152, chr3, 127414924
cg24451141, chr18, 13756072
cg20800088, chr15, 42438146
cg19026959, chr19, 13049204
cg20393002, chr3, 120909792
cg19055951, chr2, 20832097
cg27626141, chr8, 103876469
cg07786657, chr1, 167487633
cg00121148, chr3, 113603874
cg14075645, chr21, 47552343
cg08092446, chr19, 58987653
cg07508081, chr1, 161194409
cg04717777, chr1, 16090836
cg21674250, chr1, 44877746
cg24819967, chr10, 135190860
cg22501927, chr1, 21583342
cg21435360, chr12, 124586893
cg27298069, chr4, 56412099
cg10959866, chr9, 90245483
cg07973336, chr7, 23348405
cg27649346, chr18, 46303409
cg22407574, chr21, 34960583
cg05137064, chr16, 29814872
cg23247704, chr1, 116518985
cg11980897, chr1, 32237457
cg08739693, chr1, 110165726
cg05368794, chr1, 231374794
cg01065828, chr8, 86089846
cg04093159, chr5, 166211825
cg13865352, chr18, 22006621
cg05791356, chr3, 11578196
cg14867604, chr14, 100438440
cg15246232, chr2, 88991544
cg01728507, chr4, 3563357
cg24810646, chr10, 131363388
cg08639839, chr1, 149069849
cg12833948, chr16, 1384648
cg13264811, chr2, 239999466
cg18557054, chr1, 201347529
cg15578811, chr17, 79981292
cg16889168, chr20, 61474428
cg05629982, chr2, 135726792
cg11188443, chr20, 54824646
cg03681544, chr8, 144287457
cg04386130, chr3, 156209042
cg01744617, chr3, 58151629
cg21333026, chr2, 230715536
cg21221085, chr17, 16939856
cg02668075, chr2, 109558595
cg27598661, chr4, 184404666
cg22415302, chr13, 51796293
cg15709169, chr12, 125549783
cg04529955, chr4, 147442738
cg12383788, chr1, 22471748
cg05831191, chr16, 67201012
cg18634296, chr10, 25305078
cg06580782, chr3, 39093495
cg10909625, chr5, 76370281
cg16452987, chr9, 132959967
cg09047214, chr15, 101688732
cg25554740, chr10, 135047333
cg00023345, chr17, 61518492
cg00460690, chr11, 16909174
cg06818207, chr6, 6003238
cg23441673, chr11, 134831429
cg20311251, chr10, 105218160
cg24170947, chr15, 69706687
cg00656264, chr16, 87715203
cg03740761, chr20, 47237270
cg14884931, chr22, 42323359
cg25324282, chr14, 24647379
cg08331513, chr11, 2906981
cg02741216, chr10, 98945465
cg09005159, chr7, 73790765
cg09554774, chr12, 49215832
cg04828041, chr4, 40143600
cg09869051, chr1, 109289141
cg22934677, chr14, 54859664
cg10375321, chr16, 132464
cg12742320, chr14, 24033855
cg13937996, chr2, 153266209
cg22427191, chr22, 36494224
cg24930197, chr3, 41240705
cg08885316, chr17, 50487054
cg15240033, chr6, 3623985
cg06966413, chr9, 100395677
cg09796800, chr4, 128651518
cg11083745, chr5, 149494527
cg04543516, chr20, 21589093
cg19683251, chr17, 60762219
cg08743107, chr9, 91933643
cg22056112, chr13, 52707286
cg16156418, chr3, 50404342
cg19859579, chr15, 40074661
cg03698539, chr16, 68012273
cg09055236, chr7, 2673197
cg22304730, chr20, 56287294
cg08826875, chr13, 113366920
cg09291566, chr21, 44891501
cg26156154, chr12, 6587462
cg05603630, chr10, 52751341
cg20605947, chr14, 73727793
cg09590286, chr11, 45275416
cg02075410, chr4, 151504138
cg26061430, chr9, 128193128
ch.1.250064R, chr1, 6704940
cg20121920, chr12, 124833935
cg17193163, chr11, 66056724
cg15609471, chr19, 45606565
cg17773376, chr3, 107941759
cg19577984, chr14, 80669228
cg12365107, chr3, 52720035
cg10606640, chr7, 55502499
cg06322988, chr3, 138725189
cg16362030, chr22, 38136754
cg18118621, chr11, 64011997
cg22159364, chr7, 44097635
cg10811474, chr19, 8428787
cg24843474, chr1, 241520598
cg10537820, chr5, 213707
cg20938846, chr19, 50000840
cg17045801, chr7, 43153223
cg21249176, chr1, 21998094
cg25054736, chr15, 52577927
cg23710710, chr1, 40723602
cg26840672, chr6, 73867020
cg22396663, chr7, 2107835
cg21499281, chr20, 17600475
cg10188284, chr14, 64505421
cg18795727, chr14, 73065288
cg04146151, chr16, 2155961
cg25116223, chr17, 45940213
cg15148156, chr17, 33415442
cg01519223, chr6, 35351379
cg25648436, chr19, 58554527
cg21730366, chr19, 52097802
cg25995375, chr22, 38347490
cg04899896, chr4, 79860379
cg00212264, chr3, 117988595
cg10217913, chr9, 125026985
cg02789926, chr17, 48464057
cg10439889, chr22, 39105684
cg26159271, chr2, 47586936
cg14493975, chr11, 63879234
cg20435334, chr1, 1312097
cg20127188, chr22, 21996426
cg20139425, chr17, 43212881
cg18272518, chr15, 71659231
cg23023126, chr19, 308808
cg24567198, chr2, 133417634
cg08721076, chr2, 172967670
cg15383980, chr1, 113741483
cg25885449, chr18, 77638415
cg05202597, chr1, 21890260
cg05863464, chr8, 142390089
cg07134887, chr2, 234664020
cg00750074, chr16, 89608354
cg15461699, chr13, 81278703
cg13387232, chr4, 1387823
cg09325695, chr14, 37825967
cg23230362, chr11, 119060239
cg13750850, chr22, 19879377
cg06150299, chr16, 30993485
cg21103170, chr14, 101521464
cg02045416, chr2, 10355362
cg11434693, chr1, 226349343
cg04944266, chr13, 35516489
cg26904914, chr5, 39425374
cg16670155, chr19, 18557220
cg09419983, chr3, 148847273
cg20900852, chr1, 11723557
cg07124045, chr10, 103595509
cg20582655, chr1, 156390237
cg25084108, chr14, 33668205
cg04110544, chr5, 42424942
cg25794571, chr13, 29233160
cg25632986, chr16, 67229444
cg07758428, chr11, 68193549
cg07930257, chr13, 114820372
cg23807890, chr2, 228736357
cg16112558, chr17, 18157478
cg13311494, chr17, 17460905
cg14751914, chr18, 46477404
cg15788149, chr7, 116312273
cg11741902, chr3, 57113245
cg18747378, chr6, 31239247
cg01231552, chr9, 133577566
cg10853608, chr16, 713101
cg11986082, chr2, 66652819
cg16280683, chr5, 61708653
cg24862510, chr1, 164290545
cg12271199, chr1, 59248280
cg13099445, chr16, 58529666
cg10314139, chr10, 71560231
cg02547521, chr7, 44147661
cg21454030, chr4, 17812573
cg25431093, chr15, 62982653
cg16964533, chr22, 24647456
cg11697038, chr1, 27931284
cg01451391, chr17, 53343561
cg17504999, chr4, 134072723
cg25458727, chr3, 10261761
cg04901136, chr12, 46384453
cg14839905, chr11, 68183723
cg20775755, chr6, 31660252
cg03255128, chr16, 31227411
cg09012071, chr19, 2171984
cg07262320, chr5, 621696
cg27460056, chr7, 1038959
cg27051886, chr22, 42308024
cg07193511, chr11, 64017619
cg11609668, chr11, 117075078
cg04485850, chr20, 60372565
cg01640506, chr7, 150417635
cg14418275, chr11, 66243281
cg19936912, chr4, 152330012
cg15664967, chr2, 58468431
cg11692435, chr8, 1105844
cg02925268, chr12, 132301999
cg09918917, chr17, 33456630
cg03617037, chr6, 89980404
cg02318866, chr17, 74723064
cg12311052, chr3, 149687905
cg15241633, chr7, 86415945
cg26488985, chr16, 89584852
cg09583267, chr12, 132349953
cg02746308, chr3, 32827258
cg16088254, chr2, 238662099
cg14525059, chr3, 128485954
cg13323902, chr5, 140090859
cg16252905, chr1, 1150936
cg15660669, chr21, 17102764
cg18129198, chr1, 63249693
cg10161889, chr10, 30331911
cg19502932, chr11, 6640663
cg24637319, chr11, 88071195
cg11800251, chr1, 200116926
cg23452480, chr17, 43921637
cg06364063, chr20, 36795219
cg09332835, chr11, 784136
cg24159561, chr3, 32188219
cg06835772, chr16, 85296220
cg24987626, chr9, 137021144
cg18655025, chr14, 91008005
cg07279214, chr15, 22955254
cg03922095, chr7, 1525238
ch.19.36684304F, chr19, 31992464
cg05390516, chr13, 115042160
cg16191143, chr1, 25297315
cg22425466, chr9, 140115987
cg25989749, chr11, 62550245
cg18117149, chr6, 41746516
cg13625974, chr22, 20771271
cg15268826, chr4, 132897691
cg23951305, chr6, 170581890
cg26284844, chr13, 24829503
cg18346693, chr20, 62286145
cg24689021, chr11, 64043199
cg10911103, chr7, 2082116
cg07588857, chr1, 227163884
cg04373657, chr19, 2800910
cg00573191, chr3, 132246926
cg23636099, chr2, 114359251
cg08111895, chr11, 31531177
cg02347105, chr16, 21557425
cg00796030, chr11, 44608074
cg26798702, chr1, 1373044
cg11504739, chr11, 62186060
cg17436224, chr14, 23299104
cg01443832, chr7, 29725145
cg13225272, chr1, 83729633
cg01711850, chr13, 80726306
cg26729372, chr10, 131637200
cg22835523, chr15, 85668287
cg19496566, chr19, 48249018
cg02196694, chr8, 60031540
cg06984849, chr16, 2017213
cg05062179, chr17, 76987630
cg27374486, chr21, 36281988
cg25029315, chr17, 33288471
cg15495741, chr11, 78590580
cg11631579, chr5, 118309902
cg07773052, chr10, 49514483
cg21606780, chr1, 197881327
cg14250783, chr7, 157722718
cg08963419, chr19, 10402829
cg03997071, chr22, 25752084
cg16015474, chr6, 33264213
cg15320905, chr12, 114162813
cg20034440, chr10, 124717069
cg06023279, chr18, 12896925
cg23391171, chr13, 41634374
cg22054008, chr9, 131873921
cg23832749, chr10, 43608967
cg23381664, chr6, 33281712
cg24352985, chr2, 220116772
cg00904548, chr3, 49720819
cg14628323, chr17, 79906610
cg09813248, chr12, 121164194
cg26159074, chr5, 1554307
cg02469461, chr2, 231589895
cg03011594, chr16, 56370697
cg04084170, chr2, 220143147
cg15966876, chr18, 13824072
cg11924470, chr1, 151684408
cg18927317, chr7, 75987610
cg13796518, chr10, 51827657
cg27392559, chr17, 27410759
cg07381470, chr1, 1483130
cg03914014, chr19, 10420513
cg22513229, chr19, 7447757
cg06689990, chr16, 1547486
cg19713585, chr16, 2298791
cg11687406, chr20, 10199434
cg09706428, chr21, 44892086
cg02458396, chr4, 710003
cg15759988, chr8, 144717698
cg17885211, chr1, 156479602
cg12492007, chr5, 180023958
cg17512109, chr11, 124938944
cg17223541, chr4, 3228890
cg18519957, chr1, 203486002
cg11578055, chr4, 10447440
cg11196113, chr4, 13629736
cg01283246, chr5, 135266135
cg23840854, chr1, 161414152
cg17208360, chr15, 25483088
cg21179492, chr9, 138393651
cg20094085, chr10, 60936248
cg25201056, chr5, 14997275
cg10608004, chr19, 49991389
cg24025896, chr5, 87976294
cg01848616, chr3, 14191828
cg17249224, chr5, 1257061
cg19510820, chr11, 107047888
cg16530128, chr11, 2925951
cg12146100, chr16, 30712867
cg25400013, chr11, 108093439
cg20281040, chr1, 16533063
cg05557618, chr9, 136326006
cg05134567, chr19, 45449165
cg13412213, chr10, 119598364
cg08130630, chr1, 2312475
cg01496536, chr9, 140672566
cg18719814, chr4, 47916640
cg09989011, chr10, 104677906
cg13516768, chr20, 61599125
cg18532480, chr8, 66556501
cg10108042, chr16, 1808309
cg23912435, chr1, 150601613
cg09394728, chr3, 128615971
cg27264462, chr1, 2106365
cg02932840, chr10, 28591940
cg09009011, chr9, 117030773
cg05971212, chr16, 30995097
cg16576106, chr16, 4736778
cg15967278, chr8, 29886272
cg02714988, chr4, 85556351
cg13882691, chr16, 75681773
cg10034572, chr2, 160921789
cg24126199, chr20, 48771253
cg11926460, chr2, 234378272
cg01444801, chr10, 135216882
cg10902252, chr2, 27527527
cg11718315, chr15, 65579480
cg00207893, chr17, 74015638
cg26062048, chr5, 153418412
cg04203210, chr16, 2079199
cg14405625, chr9, 136855902
cg05288192, chr17, 80893564
cg18529846, chr14, 23057979
cg26401541, chr6, 91078974
cg20847581, chr19, 42746215
cg17076573, chr2, 241640172
cg17414431, chr19, 49622715
cg26635518, chr12, 110232895
cg12790440, chr16, 67464868
cg00487727, chr19, 36391260
cg10244978, chr16, 1353946
cg13735608, chr1, 110200767
cg11969813, chr17, 79816559
cg08952467, chr13, 50384748
cg15191887, chr5, 664666
cg02247881, chr19, 18599725
cg26582975, chr3, 158520111
cg00370047, chr11, 1481436
cg23496597, chr20, 57463725
cg07896133, chr16, 2060022
cg03059876, chr20, 61905353
cg00862312, chr17, 79847431
cg07415687, chr6, 7910841
cg25109721, chr17, 48125064
cg10062258, chr1, 65885702
cg08379517, chr19, 18509505
cg04588356, chr2, 3473852
cg22330533, chr22, 50173996
cg05533001, chr7, 2019608
cg21657580, chr19, 45748941
cg17704839, chr19, 9939038
cg13016048, chr9, 140054056
cg19455421, chr7, 30174596
cg07387931, chr3, 140785160
cg20968354, chr14, 104688848
cg06934003, chr12, 106871374
cg05464720, chr4, 1349298
cg15277778, chr12, 48169438
cg19343212, chr10, 73012347
cg24066474, chr11, 134104369
cg02996897, chr12, 32832074
cg14661811, chr6, 31619002
cg12084866, chr17, 39485627
cg13368085, chr19, 1470810
cg09637723, chr3, 49967744
cg07269567, chr6, 30886161
cg18178844, chr6, 32014100
cg22535104, chr7, 75300290
cg26251208, chr15, 77933917
ch.11.1112034R, chr11, 58387925
cg08982252, chr17, 73845418
cg20515846, chr10, 134605837
cg21438681, chr9, 138404917
cg15645344, chr4, 165109574
cg06459490, chr12, 124122371
cg02668843, chr17, 40575479
cg21665850, chr2, 731073
cg24814328, chr1, 32104077
cg20671801, chr12, 51041328
cg18463820, chr14, 58619219
cg21362926, chr1, 894026
cg16901014, chr8, 146051423
cg14871650, chr4, 170541668

TABLE 4
List of common DMPs within group for Batch 1 low comparisons
Probe ID, chromosome, position

cg10435849, chr21, 47518747
cg22155376, chr11, 17591837
cg18693345, chr5, 2754148
cg00848693, chr16, 69400894
cg05424199, chr16, 30759608
cg07964219, chr21, 46847898
cg06380459, chr17, 78234294
cg01558827, chr12, 89746108
cg21392845, chr17, 7227224
cg08188779, chr15, 45004577
cg21149107, chr14, 71228406
cg02884346, chr4, 6271501
cg23237353, chr4, 6712888
cg07744695, chr19, 44229909
cg07758428, chr11, 68193549
cg16362899, chr10, 121554998
cg25546329, chr19, 40175243
cg11820257, chr12, 124836411
cg17942925, chr5, 180611441
cg05512065, chr6, 92104129
cg06544141, chr1, 27320384
cg02585025, chr16, 74347151
cg17930710, chr20, 30678020
cg04929760, chr9, 97562312
cg17125331, chr16, 13861325
cg15635052, chr3, 32433105
cg16090620, chr22, 47197221
cg12163823, chr2, 242673721
cg11271430, chr4, 187984718
cg26140833, chr13, 25861650
cg18908499, chr1, 247712237
cg25927164, chr17, 17685407
cg16083044, chr6, 168788815
cg14417917, chr9, 140065568
cg13073261, chr15, 25201732
cg25106538, chr17, 5116354
cg20671801, chr12, 51041328
cg08714407, chr2, 24307071
cg07896133, chr16, 2060022
cg01728507, chr4, 3563357
cg11986082, chr2, 66652819
cg01690573, chr6, 151771312
cg24432158, chr3, 51892692
cg11349857, chr19, 2388952
cg14418275, chr11, 66243281
cg18129198, chr1, 63249693
cg27336518, chr16, 722515
cg18117149, chr6, 41746516
cg05831191, chr16, 67201012
cg27300841, chr16, 30905180
cg15268826, chr4, 132897691
cg23247704, chr1, 116518985
cg18659937, chr11, 66669653
cg11785465, chr1, 51717051
cg25972327, chr8, 94233755
cg18855351, chr13, 30920638
cg15399131, chr5, 170293065
icg23685282, chr3, 46205005
cg06754987, chr1, 44444250
cg04360557, chr1, 33282743
cg15973898, chr10, 104121704
cg14751914, chr18, 46477404
cg19131736, chr7, 1902888
cg23636099, chr2, 114359251
cg24784794, chr3, 79068831
cg09009011, chr9, 117030773
cg23807890, chr2, 228736357
cg23659696, chr6, 39083005
cg19388017, chr19, 15285100
cg02742186, chr13, 21100191
cg12365107, chr3, 52720035
cg24637319, chr11, 88071195
cg00794209, chr8, 255189
cg20393002, chr3, 120909792
cg11926460, chr2, 234378272
cg11761615, chr7, 5348924
cg11683563, chr19, 9435051
cg12271199, chr1, 59248280
cg01089702, chr8, 42552401
cg27553048, chr1, 111772613
cg07344096, chr1, 7724122
cg27380530, chr5, 173099221
cg25803700, chr11, 117233980
cg21606780, chr1, 197881327
cg12010548, chr14, 105838058
cg20979652, chr8, 40791285
cg08575696, chr12, 121087382
cg26460816, chr5, 65892187
cg02339482, chr3, 191068318
cg04819250, chr13, 111089384
cg02075410, chr4, 151504138
cg00661228, chr7, 20166735
cg03352322, chr19, 52693048
cg22678932, chr22, 22006201
cg01955286, chr3, 125475060
cg13864229, chr14, 37051830
cg23325796, chr12, 115442768
cg19539972, chr4, 7069911
cg22054008, chr9, 131873921
cg23268879, chr7, 5359951
cg05791356, chr3, 11578196
cg25231670, chr13, 68682018
cg00720322, chr17, 2154955
cg02318866, chr17, 74723064
cg00260888, chr20, 30865478
cg17251097, chr10, 81838583
cg18882457, chr6, 136247622
cg05474055, chr11, 64611972
cg18321533, chr14, 21162163
cg19496491, chr11, 12695499
cg08721076, chr2, 172967670
cg19289969, chr7, 1892989
cg12940822, chr5, 127873397
cg01263854, chr20, 54824316
cg16624187, chr3, 139108582
cg13250001, chr3, 119810597
cg01944087, chr15, 69110737
cg04110544, chr5, 42424942
cg25432518, chr7, 1894300
cg05588111, chr10, 112678594
cg10853608, chr16, 713101
cg16098051, chr17, 39663178
cg08351474, chr20, 4666916
cg00763120, chr22, 23637135
cg06753949, chr19, 15334309
cg03757430, chr11, 93269784
cg21067709, chr13, 21714627
cg18154328, chr11, 60609349
cg05321808, chr4, 187647457
cg22545649, chr4, 96470459
cg09423221, chr3, 78078946
cg17869339, chr1, 148897999
cg14696850, chr12, 7585134
cg12335414, chr10, 134359618
cg12079885, chr12, 125328475
cg10416894, chr13, 20437958
cg15709169, chr12, 125549783
cg25376334, chr3, 71429536
cg11101514, chr10, 27180907
cg19936912, chr4, 152330012
cg23441673, chr11, 134831429
cg25319564, chr13, 114214573
cg01283246, chr5, 135266135
cg12833948, chr16, 1384648
cg15006118, chr11, 78387290
cg17289308, chr11, 2588628
cg15524632, chr19, 19117770
cg05736120, chr6, 170125165
cg22680823, chr18, 56459606
cg13823617, chr1, 6421969
cg09179999, chr15, 77811681
cg07818050, chr5, 134685323
cg24938752, chr3, 128294769
cg13514165, chr9, 130544369
cg27464104, chr16, 67926805
cg15635599, chr9, 97020433
cg15047751, chr3, 52395045
cg04640920, chr15, 37180889
cg23827795, chr12, 3736446
cg18864497, chr6, 7313384
cg13724220, chr2, 3383413
cg14322961, chr9, 91789383
cg06533586, chr3, 99630775
cg03611733, chr15, 45585637
cg12197666, chr13, 40511553
cg05276408, chr10, 1994537
cg16112558, chr17, 18157478
ch.6.609614F, chr6, 24693000
cg19086321, chr15, 57842332
cg12499716, chr19, 30096869
cg22425466, chr9, 140115987
cg12078605, chr2, 80531500
cg23526353, chr4, 100484905
cg26079579, chr1, 33896226
cg04731570, chr5, 139487691
cg19524023, chr6, 170592108
cg08700690, chr15, 60884630
cg09325695, chr14, 37825967
cg00633933, chr22, 50280082
cg04551967, chr17, 1837764
cg10811474, chr19, 8428787
cg26163843, chr14, 34944819
cg21573582, chr11, 77300763
cg25458727, chr3, 10261761
cg06485995, chr7, 27547647
cg23023126, chr19, 308808
cg27106230, chr13, 99229068
cg05870631, chr17, 20450466
cg08987887, chr6, 76311629
cg16335841, chr2, 233246214
cg24549058, chr14, 70006946
cg20892288, chr9, 128003619
cg12194824, chr19, 5691122
cg11578055, chr4, 10447440
cg09788788, chr4, 87468529
cg26201787, chr12, 77157648
cg21594328, chr2, 206830726
cg01429408, chr1, 156860314

1. A method of identifying oxidative stress (OS) in a test cell, comprising
(a) determining the methylation status of at least one CpG site in a DNA sample obtained from the test cell,
(b) comparing the methylation status of the CpG site from (a) with that of a control without OS,
wherein difference in the methylation status of the CpG site in the test cell compared to the CpG site in the control is indicative of the test cell having OS.

2. The method according to claim 1, wherein the difference in methylation status is hypomethylation or hypermethylation of the CpG site in the test cell and the hypomethylation or hypermethylation of the CpG site is indicative of OS in the test cell.

3. The method according to claim 1, wherein the CpG site is selected from the list of CpG sites in the table below:
Probe ID, chromosome, position
cg10435849, chr21, 47518747
cg11349857, chr19, 2388952
cg00848693, chr16, 69400894
cg21149107, chr14, 71228406
cg02585025, chr16, 74347151
cg18693345, chr5, 2754148
cg25126321, chr11, 66462313
cg22155376, chr11, 17591837
cg17251097, chr10, 81838583
cg16098051, chr17, 39663178
cg23153556, chr11, 64900163
cg11271430, chr4, 187984718
cg22545649, chr4, 96470459
cg05736120, chr6, 170125165
cg14417917, chr9, 140065568
cg23526353, chr4, 100484905
cg02884346, chr4, 6271501
cg12528713, chr4, 81187605
cg05943476, chr2, 70780904
cg03379894, chr5, 141131868
cg17135325, chr3, 160939158
cg16362899, chr10, 121554998
cg25546329, chr19, 40175243
cg00532451, chr1, 228528913
cg02339482, chr3, 191068318
cg01955286, chr3, 125475060
cg23268879, chr7, 5359951
cg01944087, chr15, 69110737
cg06544141, chr1, 27320384
cg20076186, chr3, 128372563
cg20906710, chr12, 50065474
cg25319564, chr13, 114214573
cg21598631, chr2, 242154679
cg20393002, chr3, 120909792
cg27626141, chr8, 103876469
cg14075645, chr21, 47552343
cg08351474, chr20, 4666916
cg15047751, chr3, 52395045
cg23247704, chr1, 116518985
cg05791356, chr3, 11578196
cg01728507, chr4, 3563357
cg12833948, chr16, 1384648
cg08714407, chr2, 24307071
cg18908499, chr1, 247712237
cg15709169, chr12, 125549783
cg12383788, chr1, 22471748
cg05831191, chr16, 67201012
cg06818207, chr6, 6003238
cg23441673, chr11, 134831429
cg11785465, chr1, 51717051
cg01429408, chr1, 156860314
cg02075410, chr4, 151504138
cg17773376, chr3, 107941759
cg12365107, chr3, 52720035
cg23023126, chr19, 308808
cg08721076, chr2, 172967670
cg09325695, chr14, 37825967
cg11434693, chr1, 226349343
cg04110544, chr5, 42424942
cg07758428, chr11, 68193549
cg10811474, chr19, 8428787
cg23807890, chr2, 228736357
cg16112558, chr17, 18157478
cg14751914, chr18, 46477404
cg10853608, chr16, 713101
cg11986082, chr2, 66652819
cg12271199, chr1, 59248280
cg25458727, chr3, 10261761
cg14418275, chr11, 66243281
cg19936912, chr4, 152330012
cg02318866, chr17, 74723064
cg18129198, chr1, 63249693
cg24637319, chr11, 88071195
cg09332835, chr11, 784136
cg22425466, chr9, 140115987
cg18117149, chr6, 41746516
cg15268826, chr4, 132897691
cg23636099, chr2, 114359251
cg09788788, chr4, 87468529
cg26163843, chr14, 34944819
cg13864229, chr14, 37051830
cg17942925, chr5, 180611441
cg18659937, chr11, 66669653
cg03611733, chr15, 45585637
cg13514165, chr9, 130544369
cg04360557, chr1, 33282743
cg11578055, chr4, 10447440
cg01283246, chr5, 135266135
cg21606780, chr1, 197881327
cg09009011, chr9, 117030773
cg11926460, chr2, 234378272
cg11718315, chr15, 65579480
cg22054008, chr9, 131873921
cg07896133, chr16, 2060022
cg25109721, chr17, 48125064
cg20671801, chr12, 51041328

4. The method according to claim 1, wherein in step (a) the methylation status of at least 2 CpG sites are determined.

5. The method according to claim 1, wherein in step (a) the methylation status of at least 3 CpG sites are determined.

6. The method according to claim 1, wherein in step (a) the methylation status of at least 5 CpG sites are determined.

7. The method according to claim 3, wherein in step (a) the methylation status of all the CpG sites listed is determined.

8. The method according to claim 1, further comprising the step of:
(i) performing bisulfite modification to the DNA sample before step (a).

9. The method according to claim 1, wherein the cell is obtained from a biological sample selected from the group consisting of blood, brain, sperm and any other tissue or sample that provides genomic DNA.

10. The method according to claim 1 wherein the cell is a eukaryote.

11. The method according to claim 1, wherein the cell is from a mammal.

12. The method according to claim 11, wherein the mammal is a mouse, a rat, a guinea pig, a dog, a mini-pig, a human being, a cow, a sheep, a pig, a goat, a horse, a donkey, and a mule.

13. The method according to claim 1, wherein the cell is a skin cell, a stem cell or a cell derived therefrom.
